(19) United States
(12) Patent Application Publication
(10) Pub. No.: US 2025/0011875 A1
(43) Pub. Date: Jan. 9, 2025

(54) METHOD AND BIOMARKERS FOR ASSESSING THE RISK AND PROGNOSTIC LEVEL OF METASTATIC COLORECTAL CANCER

(71) Applicants: CHANG GUNG UNIVERSITY, Taoyuan City (TW); CHANG GUNG MEMORIAL HOSPITAL, LINKOU, TAOYUAN CITY (TW)

(72) Inventors: Hsuan Liu, Taoyuan City (TW); Hung-Chi Hsu, Taoyuan City (TW); Chan-Keng Yang, Taoyuan City (TW)

(21) Appl. No.: 18/669,240

(22) Filed: May 20, 2024

(30) Foreign Application Priority Data
Jul. 5, 2023 (TW) 112125120

(51) Int. Cl. C12Q 1/6886

(52) U.S. Cl. CPC C12Q 1/6886; C12Q 2600/118; C12Q 2600/158; C12Q 2600/178

(57) ABSTRACT

This invention unveils a method and biomarkers for assessing the risk and prognostic level of metastatic colorectal cancer. The evaluation is made by observing the expression levels of EV-miR-320c in extracellular vesicles (EV), identified as the biomarker in this invention. This measure helps determine the risk of an individual developing metastatic colorectal cancer. Furthermore, post-treatment, the expression levels of EV-miR-320c in EVs can be used to monitor the progression of the disease. The detection and monitoring methodology leveraging EV-miR-320c in EVs demonstrated high sensitivity and specificity.

### FIELD OF THE INVENTION

The present invention relates to a method and biomarkers for assessing the risk and prognostic level of cancer, especially to a method and biomarkers for assessing the risk and prognostic level of metastatic colorectal cancer.

### BACKGROUND OF THE INVENTION

Colorectal cancer, also known as large bowel cancer, rectal cancer, colon cancer, or bowel cancer, etc., is a cancer originating from the colon or rectum (which is a part of the large intestine). It occurs when cells in the colon or rectum grow abnormally and can invade or spread to other parts of the body. Symptoms include blood in the stool, changes in bowel habits, weight loss, and feeling tired.

Most colorectal cancers are caused by lifestyle habits and aging, while a small number of colorectal cancers are caused by genetic diseases. The main risk factors include diet (red meat, processed meat, alcohol, etc.), obesity, smoking, and lack of exercise.

Other risk factors include inflammatory bowel disease (divided into Crohn's disease and ulcerative colitis), familial adenomatous polyps, etc. However, these genetic diseases account for less than 5% of all cases of colorectal cancer. It is mainly a matter of living habits.

The staging of colorectal cancer is basically based on the depth of tumor invasion into the intestinal mucosa, the presence or absence of lymph node invasion, and the presence or absence of distant metastasis. The first- and second-stage cancer cells are limited to the intestine. The third-stage cancer cells will further invade the lymph nodes near the intestine. The fourth-stage cancer cells spread from the intestine to other organs, such as the liver or lungs. The fourth stage is characterized by distant metastatic colorectal cancer.

Metastatic colorectal cancer is one of the leading causes of malignancy-related death worldwide and carries a poorer survival prognosis than other cancers. The current clinical treatment strategy for metastatic colorectal cancer is a combination of chemotherapy and targeted therapy (such as Bevacizumab, Cetuximab, Panitumumab, etc.). For this reason, it is necessary to regularly observe the treatment effectiveness and prognostic level before adjusting the therapy.

Although more active drugs (such as 5-Fu, Capecitabine, Irinotecan, and Oxaliplatin, etc.) are available to help improve the prognosis of patients with metastatic colorectal cancer, drug resistance has become a limiting factor in successful cure.

Therefore, prevention is better than cure. If the risk of metastasis of the patient can be detected early, complete surgical resection may be possible, and the five-year survival rate can be improved while maintaining a certain quality of life as well as extending life.

Accordingly, developing methods and biomarkers that can inspect the risk of metastatic colorectal cancer and at the same time accurately assess/monitor cancer efficacy, cancer recurrence, and cancer metastasis are issues that those skilled in the art want to solve.

### SUMMARY OF THE INVENTION

The main objective of the present invention is to provide a method and biomarkers for assessing the risk of metastatic colorectal cancer. The evaluation is made by observing the expression levels of EV-miR-320c in extracellular vesicles (taken from biological samples), identified as the biomarker. This measure (evaluation by observing the expression levels of EV-miR-320c) helps determine the risk of an individual developing metastatic colorectal cancer.

A further objective of this invention is to provide a method and biomarkers for assessing the prognosis and evaluating the treatment efficacy of metastatic colorectal cancer. Utilizing EV-miR-320c in extracellular vesicles from biological samples as the biomarker, this approach allows for the determination of the patient's prognosis and the assessment of treatment effectiveness based on the expression levels of EV-miR-320c.

To achieve the main objective, the present invention discloses a method for assessing the risk of metastatic colorectal cancer, which comprises steps of: separating a plurality of extracellular vesicles from a biological sample of an inspection subject; using an inspection method to inspect the expression level of a biomarker in the plurality of extracellular vesicles; and judging that the inspection subject has the risk of metastatic colorectal cancer when the expression level of the biomarker is a multiple of a standard expression level. In particular, the multiple is between 6 and 8; and the biomarker is EV-miR-320c with the sequence of SED ID NO. 1.

According to an embodiment of the present invention, in the step of separating a plurality of extracellular vesicles from a biological sample of an inspection subject, the biological sample is taken from blood, plasma, or serum.

According to an embodiment of the present invention, in the step of using an inspection method to inspect the expression level of a biomarker in the plurality of extracellular vesicles, the inspection method is a real-time quantitative reverse transcription polymerase chain reaction (qRT-PCR).

According to an embodiment of the present invention, in the step of judging that the inspection subject has the risk of metastatic colorectal cancer when the expression level of the biomarker is a multiple of a standard expression level, the standard expression level is taken from a healthy sample.

Furthermore, the present invention also discloses a biomarker used for accessing the risk of metastatic colorectal cancer. The biomarker is taken from a plurality of extracellular vesicles and is EV-miR-320c with the sequence of SED ID NO. 1.

In addition to assessing the risk of metastatic colorectal cancer, another objective of the present invention is to provide a method and biomarkers for assessing the prognostic level of metastatic colorectal cancer. The evaluation is made by observing the expression levels of EV-miR-320c in extracellular vesicles (taken from biological samples), identified as the biomarker in this invention. This measure (evaluation by observing the expression levels of EV-miR-320c) helps determine the prognostic level of an individual.

To achieve the objective, the present invention discloses a method for assessing the prognostic level of metastatic colorectal cancer, which comprises steps of: separating a plurality of extracellular vesicles from a biological sample of an inspection subject; using an inspection method to inspect the expression level of a biomarker in the plurality of extracellular vesicles; and judging the prognostic level of the inspection subject according to the expression level of the biomarker. In particular, the biomarker is EV-miR-320c with the sequence of SED ID NO. 1; and the expression level of the biomarker and the prognostic level are positively correlated.

According to an embodiment of the present invention, in the step of separating a plurality of extracellular vesicles from a biological sample of an inspection subject, the biological sample is taken from blood, plasma, or serum.

According to an embodiment of the present invention, in the step of separating a plurality of extracellular vesicles from a biological sample of an inspection subject, the inspection subject has received treatment for metastatic colorectal cancer, in which the treatment for metastatic colorectal cancer is selected from the group consisting of surgery, radiotherapy, chemotherapy, targeted therapy, and thermal therapy.

According to an embodiment of the present invention, in the step of using an inspection method to inspect the expression level of a biomarker in the plurality of extracellular vesicles, the inspection method is a real-time quantitative reverse transcription polymerase chain reaction (qRT-PCR).

Furthermore, the present invention also discloses a biomarker used for assessing the prognostic level of metastatic colorectal cancer. The biomarker is taken from a plurality of extracellular vesicles and is EV-miR-320c with the sequence of SED ID NO. 1.

### BRIEF DESCRIPTION OF DRAWINGS

FIG. 1 shows a flowchart according to the first embodiment of the present invention;

FIG. 2A shows a chart of relative expression level according to the first embodiment of the present invention;

FIG. 2B shows a diagram of the ROC curve according to the first embodiment of the present invention;

FIG. 3 shows a flowchart according to the second embodiment of the present invention;

FIG. 4A shows a correlation chart between EV-miR-320c and CEA according to the present invention;

FIG. 4B shows a correlation chart between EV-miR-320c and tumor size according to the present invention;

FIG. 4C shows a correlation chart between CEA and tumor size according to the present invention;

FIG. 4D shows a correlation curve between EV-miR-320c and tumor size according to the present invention; and

FIG. 5 shows a relative expression chart of EV-miR-320c and cancer staging according to the present invention.

### DETAILED DESCRIPTION OF THE INVENTION

In view of the fact that once colorectal cancer metastasizes, the survival rate of patients will drop significantly. Therefore, it is urgent to know in advance whether patients are at risk of metastatic colorectal cancer. Accordingly, the present invention proposes method and biomarkers for assessing the risk and prognostic level of metastatic colorectal cancer to solve the problems encountered in the prior art.

In the following, the properties, the structures, and the method according to the present invention will be further described.

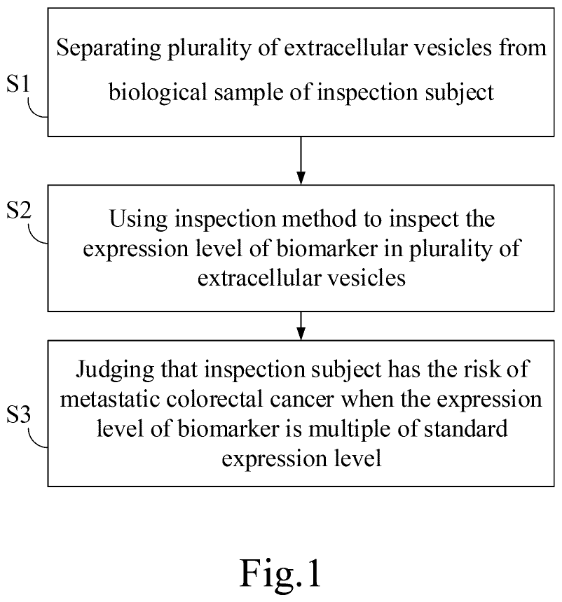

Please refer to FIG. 1, which shows a flowchart according to the first embodiment of the present invention. As shown in FIG. 1, the present invention provides a method for assessing the risk of metastatic colorectal cancer, which comprises steps of:
S1: Separating plurality of extracellular vesicles from biological sample of inspection subject;
S2: Using inspection method to inspect the expression level of biomarker in plurality of extracellular vesicles; and
S3: Judging that inspection subject has the risk of metastatic colorectal cancer when the expression level of biomarker is multiple of standard expression level.

As shown in the step S1, separate a plurality of extracellular vesicles (EV) from a biological sample of an inspection subject. According to the first embodiment of the present invention, it is used in general basic health examinations (self-pay or health insurance) to understand whether there is the risk of suffering from metastatic colorectal cancer, or whether there is the risk of metastasis in the case of colorectal cancer (transformed into metastatic colorectal cancer).

According to the first embodiment of the present invention, the inspection subject is a person in need of general health examination, a patient who has already suffered from colorectal cancer, or a patient with a history of colorectal cancer (family history). The biological sample is taken from blood, plasma, or serum. The biological sample is prepared by collecting 2 mL of fresh blood in a blood collection tube (containing EDTA, which has been stored at 4° C. for 4 hours) and centrifuging at 1900×g for 10 minutes at 4° C. The plasma (yellow) was subsequently transferred to a new tube, followed by additional centrifugation at 3000×g and stored in aliquots at −80° C.

Through the above two-step centrifugation, the influence of cell debris is minimized as much as possible.

Next, according to the relevant steps in the exoRNeasy serum/plasma handbook of QIAGEN, the plurality of extracellular vesicles is separated from the biological sample. When the plurality of extracellular vesicles are bound to the exoEasy membrane affinity column (this can solve the problem of low recovery rate and maintaining high purity), QIAzo1 was added to the spin column to cleave the plurality of extracellular vesicles and further collected by centrifugation. After extraction with chloroform, the sample was thoroughly mixed and centrifuged to separate the organic and aqueous phases. The aqueous phase is further recycled and mixed with ethanol, then added to the RNeasy MinElute spin column and centrifuged. The RNeasy MinElute spin column is then washed once with buffer (RWT) and then twice with buffer (RPE), and RNA is eluted. Subsequently, the RNA Nano 6000 detection kit of the Agilent Bioanalyzer 2100 system (Agilent Technologies, CA, USA) was used to evaluate the concentration, purity, and integrity of the RNA in the plurality of extracellular vesicles.

As shown in the step S2, use an inspection method to inspect the expression level of a biomarker in the plurality of extracellular vesicles. The biomarker according to the present invention is EV-miR-320c, which is the microRNA 320c in the extracellular vesicles with the sequence of AAAAGCUGGGUUGAGAGGGU (as shown in SED ID NO. 1). The inspection method is a real-time quantitative reverse transcription polymerase chain reaction (qRT-PCR).

Regarding the miRNA reverse transcription reaction, the total RNA in 1 g cells (HT-29 or HCT116 colorectal cancer cells) or 150 ng EV RNA taken from the biological sample is first converted into cDNA by means of Superscript III Invitrogen.

For real-time quantification of cDNA (reverse transcribed from miRNA), real-time quantitative polymerase chain reaction (real-time PCR) is performed using the standard SYBR green method on an Applied Biosystems 7500 Fast PCR System (Foster City, CA, USA). For quantitative result analysis, the Ct value of the miRNA must be normalized to the U6 gene in the same sample. The obtained Ct can be further used to evaluate the relative gene expression between different experimental groups (compared to the control group).

In order to obtain the biomarker of the present invention (EV-miR-320c, which is microRNA 320c in the extracellular vesicles), a miRNA-specific stem-loop reverse transcription primer was used during reverse transcription with the following sequence: miR-320c: CTCAACTGGTGTCGTGGAGTCGGCAATCAGTTGAGACCCTCTCAAC (SED ID NO. 2) and U6: CTCAACTGGTGTCGTGGAGTCGGCAATTCAGTTGAGAAAATATGGG (SED ID NO. 3).

In the real-time quantitative part, the primer sequences used are as follows: miR-320c: CGGCGGAAAAGCTGGGTTGAGAG (SED ID NO. 4); U6: CAAATTCGTGAAGCGTTCCA (SED ID NO. 5); universal reverse primer: CAACTGGTGTCGTGGAGTCGG (SED ID NO. 6).

Finally, as shown in the step S3, judge that the inspection subject has the risk of metastatic colorectal cancer when the expression level of the biomarker (EV-miR-320c) is a multiple of a standard expression level. The standard expression level is obtained from EV-miR-320c in a healthy sample (from a healthy person). The acquisition, separating, reverse transcription, and quantification of the standard expression level are the same as the above-mentioned method. The multiple is between 6 and 8 (preferably 6.29).

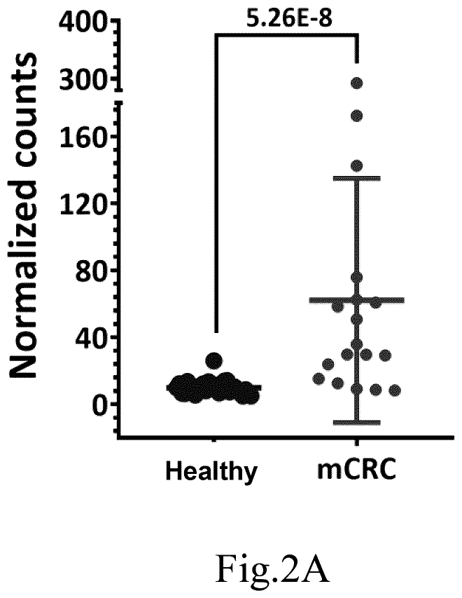

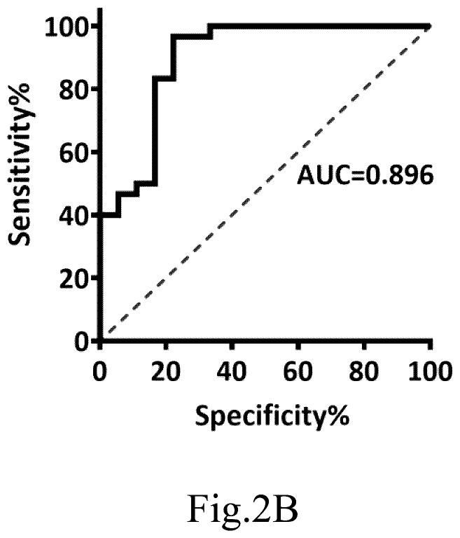

Moreover, confirmation was conducted on 18 patients with metastatic colorectal cancer at different stages and 30 healthy subjects. The results are shown in FIGS. 2A and 2B, which show a chart of relative expression level and a diagram of the ROC curve, respectively, according to the first embodiment of the present invention. According to FIGS. 2A and 2B, it is observed that the expression level of the biomarker (EV-miR-320c) in patients with metastatic colorectal cancer (mCRC) at different stages is higher than that in healthy subjects. When the multiple is 6.29, the AUC value is 0.896. The results were analyzed using ROC curve and Pearson correlation coefficient analysis.

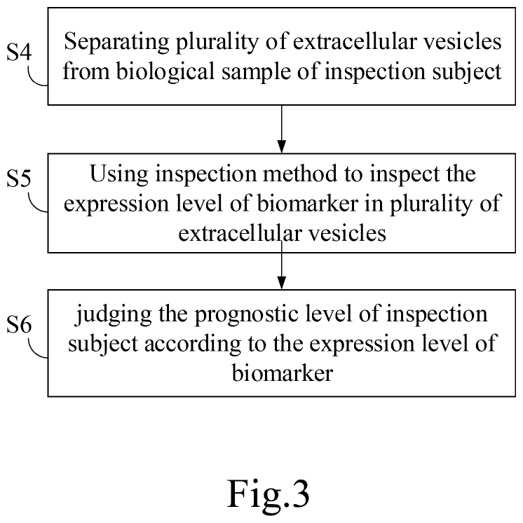

Next, please refer to FIG. 3, which shows a flowchart according to the second embodiment of the present invention. As shown in FIG. 3, the present invention provides a method for assessing the prognostic level of metastatic colorectal cancer, which comprises steps of:
S4: Separating plurality of extracellular vesicles from biological sample of inspection subject;
S5: Using inspection method to inspect the expression level of biomarker in plurality of extracellular vesicles; and
S6: Judging the prognostic level of inspection subject according to the expression level of biomarker.

As shown in the step S4, the steps for preparing the biological sample and separating the plurality of extracellular vesicles are the same as those in the first embodiment (the biological sample is also taken from blood, plasma, and serum). However, the second embodiment of the present invention is used in the follow-up tracking of cancer, so as to monitor the tumor during subsequent treatment to understand the prognostic level after treatment and correct the course of treatment. For this reason, the inspection subject of the second embodiment is a patient who has received treatment for metastatic colorectal cancer, wherein the treatment for metastatic colorectal cancer is selected from the group consisting of surgery, radiotherapy, chemotherapy, targeted therapy, and heat therapy.

Next, the inspection method and the real-time quantitative reverse transcription in the step S5 according to the second embodiment are identical to those in the step S2 according to the first embodiment. The biomarker of the plurality of extracellular vesicles is EV-miR-320c, which is the microRNA 320c in the extracellular vesicles. Likewise, the inspection method is a real-time quantitative reverse transcription polymerase chain reaction (qRT-PCR).

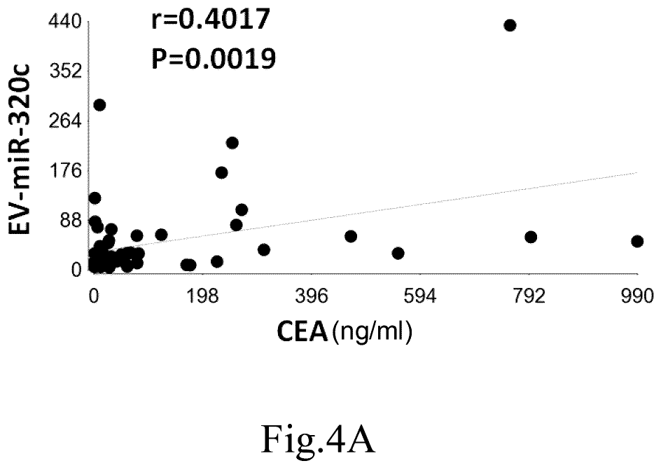

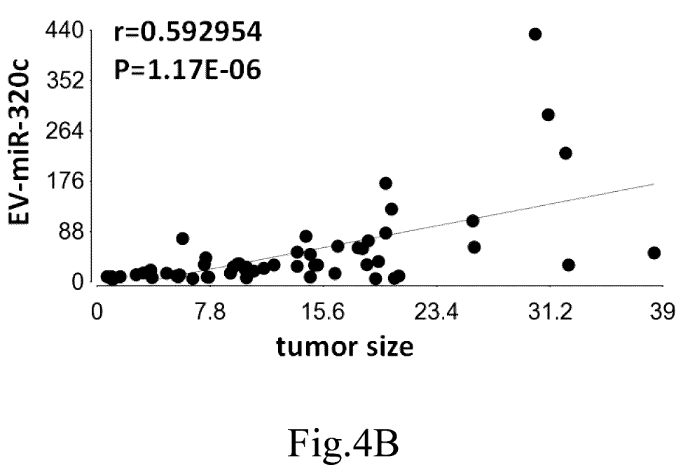

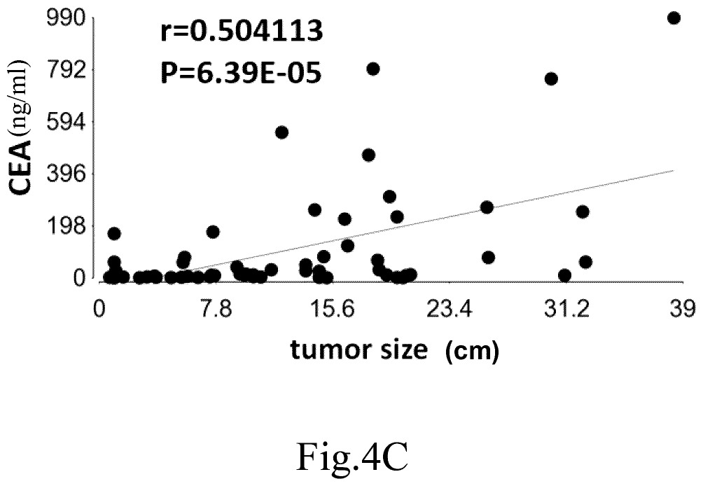

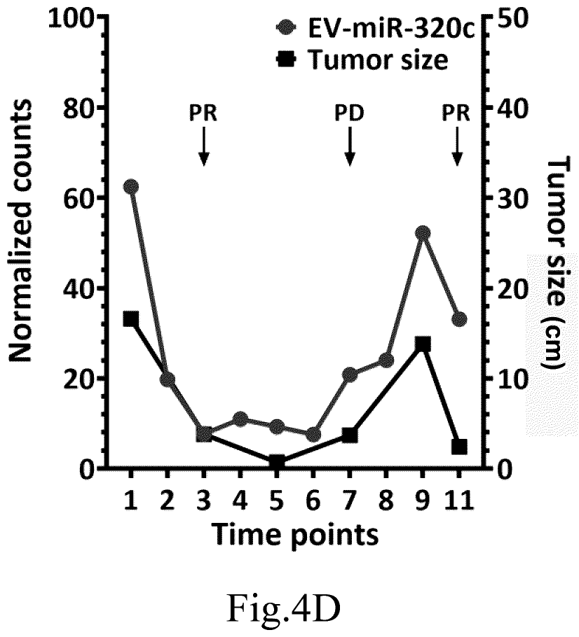

Finally, as shown in the step S6, judge the prognostic level of the inspection subject according to the expression level of the biomarker. The expression level of the biomarker and the prognostic level are positively correlated. Besides, please refer to FIG. 4A, which shows a correlation chart between EV-miR-320c and CEA according to the present invention.

Currently, CEA (Carcinoembryonic Antigen) is the most widely used molecular marker for metastatic colorectal cancer (mCRC) in blood. There are two biggest uses of CEA in clinical practice: 1. To evaluate the effectiveness of surgery or other treatments, such as whether the condition is stabilizing or may relapse. 2. Assess the possibility of cancer metastasis.

In order to further confirm the correlation between EV-miR-320c and CEA, the present invention inspects EV-miR-320c (normalized counts) and the expression level of CEA (ng/ml) in patients with colorectal cancer at different stages. The results are shown in FIG. 4A. It can be seen that EV-miR-320c and CEA show a moderate correlation (the results were analyzed using Pearson correlation coefficient analysis), showing that EV-miR-320c has the potential of acting as the biomarker for metastatic colorectal cancer (mCRC).

Next, please refer to FIGS. 4B, 4C, and 4D. FIG. 4B shows a correlation chart between EV-miR-320c and tumor size according to the present invention; FIG. 4C shows a correlation chart between CEA and tumor size according to the present invention; and FIG. 4D shows a correlation curve between EV-miR-320c and tumor size according to the present invention. As shown in FIG. 4B, there is a significant positive correlation between the expression level of EV-miR-320c and tumor size (cm, the tumor measurement method is computed tomography), with high efficiency (r=0.592954, p=1.17E-06). Furthermore, as shown in FIG. 4C, there is also a significant positive correlation between the expression level of CEA (ng/ml) and tumor size (cm, the tumor measurement method is computerized tomography) (r=0.504113, p=6.39E-05).

Comparing FIG. 4B and FIG. 4C, it can be seen that compared with the CEA inspection according to the prior art, EV-miR-320c of the present invention has a higher and better correlation with tumor size, showing that it is more effective in metastatic colorectal cancer (mCRC). In terms of biomarkers, EV-miR-320c of the present invention has more advantages than CEA.

Next, taking mCRC patient 5 (CRC005) as a representative example, it can be seen from FIG. 4D that the expression level (normalized counts) of EV-miR-320c is correlated with the change in tumor size (cm) of mCRC patient 5 (CRC005) (the time points are 4-6 weeks apart). For example, the expression level of EV-miR-320c was significantly reduced during the initial partial response to first-line treatment (at this time, it is a PR state. Partial response (PR): refers to a significant reduction in tumor size). When mCRC patients 5 relapses (at this time, it is a PD state. Progressive disease (PD): refers to the situation when the tumor is found to become larger; the tumor (metastasis) is also found in other organs and tissues; the cancer indicators increase abnormally; and the patient's symptoms become more severe), the expression level of EV-miR-320c was increased accordingly. Then after switching to second-line treatment (returning to PR status), the expression level of EV-miR-320c dropped significantly again.

Accordingly, from the moderate correlation with CEA and the correlation with tumor size, it can be known that EV-miR-320c of the present invention can become a powerful indicator of various clinical attributes, such as the detection of metastatic colorectal cancer and real-time monitoring of treatment response.

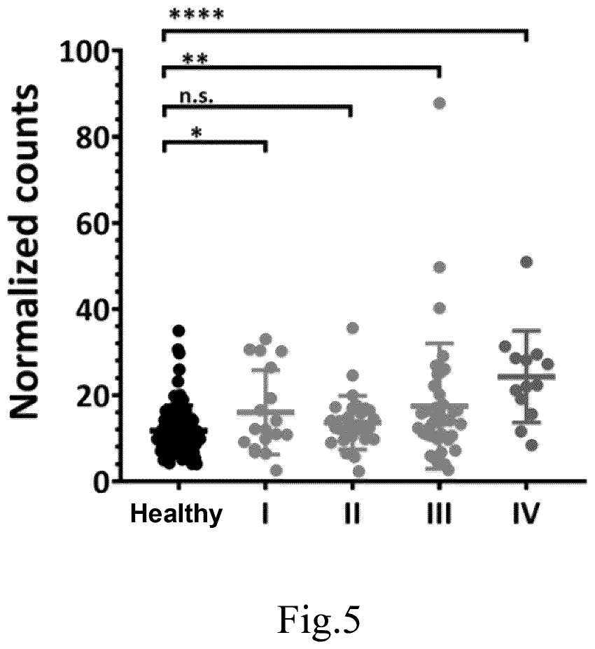

Moreover, 102 patients with metastatic colorectal cancer (mCRC) at different stages (I, II, III, IV) and 90 healthy subjects (Healthy) were used to measure the expression level and correlation of EV-miR-320c (normalized counts). The results are shown in FIG. 5. Compared with healthy subjects, the expression level of EV-miR-320c is significantly higher in mCRC late-stage (IV), showing that EV-miR-320c of the present invention can be used as a biomarker for staging metastatic colorectal cancer.

It can be known that EV-miR-320c of the present invention can be used as a biomarker of metastatic colorectal cancer. Whether a person has already suffered from colorectal cancer or has a history of colorectal cancer, it can also be used as a daily health check (like common tumor markers). EV-miR-320c can be used to determine whether there is the risk of metastatic colorectal cancer from a simple and small number of biological samples, thereby reducing the occurrence of late detection of metastatic colorectal cancer.

Furthermore, EV-miR-320c according to the present invention can also be used to inspect the staging of metastatic colorectal cancer and real-time monitoring of treatment response. With a simple and small number of biological samples, the current patient's condition can be known in real time, allowing doctors to track and modify treatment methods during the treatment process to improve the patient's survival rate.

1. A method for assessing the risk of metastatic colorectal cancer, comprising steps of:
separating a plurality of extracellular vesicles from a biological sample of an inspection subject;
using an inspection method to inspect the expression level of a biomarker in the plurality of extracellular vesicles; and
judging that the inspection subject has the risk of metastatic colorectal cancer when the expression level of the biomarker is a multiple of a standard expression level;
wherein the multiple is between 6 and 8; and the biomarker is EV-miR-320c with the sequence of SED ID NO. 1.

2. The method for assessing the risk of metastatic colorectal cancer of claim 1, where in the step of separating a plurality of extracellular vesicles from a biological sample of an inspection subject, the biological sample is taken from blood, plasma, or serum.

3. The method for assessing the risk of metastatic colorectal cancer of claim 1, where in the step of using an inspection method to inspect the expression level of a biomarker in the plurality of extracellular vesicles, the inspection method is a real-time quantitative reverse transcription polymerase chain reaction (qRT-PCR).

4. The method for assessing the risk of metastatic colorectal cancer of claim 1, where in the step of judging that the inspection subject has the risk of metastatic colorectal cancer when the expression level of the biomarker is a multiple of a standard expression level, the standard expression level is taken from a healthy sample.

5. A method for assessing the prognostic level of metastatic colorectal cancer, comprising steps of:
separating a plurality of extracellular vesicles from a biological sample of an inspection subject;
using an inspection method to inspect the expression level of a biomarker in the plurality of extracellular vesicles; and
judging the prognostic level of the inspection subject according to the expression level of the biomarker;
wherein the biomarker is EV-miR-320c with the sequence of SED ID NO. 1; and the expression level of the biomarker and the prognostic level are positively correlated.

6. The method for assessing the prognostic level of metastatic colorectal cancer of claim 5, where in the step of separating a plurality of extracellular vesicles from a biological sample of an inspection subject, the biological sample is taken from blood, plasma, or serum.

7. The method for assessing the prognostic level of metastatic colorectal cancer of claim 5, where in the step of separating a plurality of extracellular vesicles from a biological sample of an inspection subject, the inspection subject has received treatment for metastatic colorectal cancer, in which the treatment for metastatic colorectal cancer is selected from the group consisting of surgery, radiotherapy, chemotherapy, targeted therapy, and thermal therapy.

8. The method for assessing the prognostic level of metastatic colorectal cancer of claim 5, where in the step of using an inspection method to inspect the expression level of a biomarker in the plurality of extracellular vesicles, the inspection method is a real-time quantitative reverse transcription polymerase chain reaction (qRT-PCR).

9. A biomarker used for accessing the risk of metastatic colorectal cancer, taken from a plurality of extracellular vesicles, and being EV-miR-320c with the sequence of SED ID NO. 1.

10. A biomarker used for assessing the prognostic level of metastatic colorectal cancer, taken from a plurality of extracellular vesicles, and being EV-miR-320c with the sequence of SED ID NO. 1.
